(19) United States
(12) Patent Application Publication
(10) Pub. No.: US 2003/0068347 A1
(43) Pub. Date: Apr. 10, 2003

(54) Use of microbially encapsulated meterials in cosmetic end formulations

(76) Inventors: Werner Baschong, Basel (CH); Dietmar Huglin, Eimeldingen (DE)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/169,700

(22) PCT Filed: Jan. 4, 2001
(86) PCT No.: PCT/EP01/00051

(30) Foreign Application Priority Data
Jan. 10, 2000 (EP) 00810021.6

(51) Int. Cl. A61K 6/00

(52) U.S. Cl. 424/401

(57) ABSTRACT

The use of microbially encapsulated materials (filled biocapsules), in particular yeast, in cosmetic end formulations is described.

The present invention relates to the use of microbially encapsulated materials in cosmetic end formulations, to cosmetic end formulations comprising these biocapsules, and to the various uses of these end formulations.

It is known to encapsulate various active ingredients into microcapsules which occur naturally or which may otherwise be referred to as biocapsules.

EP-A-0,242,135 describes the general process of encapsulation, in particular yeast capsules.

WO 94/22572 discloses an improved preparation method of biocapsules, and preferred uses, for example food additives.

Surprisingly, it has now been found that microbially encapsulated cosmetic active ingredients can be incorporated into cosmetic end formulations in a very simple manner while safeguarding their morphological and physicochemical properties.

Accordingly, the present invention provides for the use of microbially encapsulated materials in cosmetic end formulations. The use according to the invention is notable for the fact that microbially encapsulated material comprises one or more liquid or microbially encapsulatable cosmetic active ingredients.

The microbial material which is used for the encapsulation is preferably a fungus. Typical examples are yeasts, for example Saccharomyces cerevisiae (brewer's yeast and baker's yeast), Kluyveromyces fragilis (dairy yeast), and Candida utilis, and fibrous fungi, for example Aspergillus niger.

Further microbial materials which can be used according to the invention are bacteria and algae.

According to the invention, preference is given to using yeast as the microbial material.

According to the invention, the microbial material is in fully grown form, i.e. it has been harvested from its culture medium and is intact. The microbial material is preferably living, at least at the start of the incorporation process of the cosmetic active ingredients.

The microbial material preferably has a cell diameter of about 5 μm. Bacteria can have a smaller cell size of about 1 to 2 μm and be cultivated for a relatively large cell diameter.

For the present invention, it is not necessary for the microbial material to have a certain lipid content. Typically, the lipid content is not more than 5%, preferably up to 3%, of the dry weight of the microbe.

The material to be encapsulated, i.e. the cosmetic active ingredient, should be in liquid form throughout the treatment. Here, the material may itself be liquid (including oil), or it is also possible to use a solid which is dissolved or microdispersed in a suitable solvent or dispersant. In this connection, it is important that a solvent is chosen which is not also miscible with the microbial material. Examples of suitable solvents are lower alcohols, for example methanol, ethanol or isopropanol. The solvent may, if desired, be removed following the encapsulation process, for example by spray drying.

The material to be encapsulated must not be dissolved in any lipid-forming part of the microbial material and should not cause tearing of the microbial cell wall.

Suitable encapsulatable materials are an active ingredient suitable for skin cosmetics, an active ingredient composition or an active ingredient extract, one ingredient or a mixture of ingredients approved for dermal or topical administration.

By way of example, the following may be listed:

active ingredients which have a cleansing and care action on the surface of the skin and the hair. These include all substances used for skin cleansing, such as oils, soaps, syndets and solid substances;

active ingredients with a deodorizing, antiperspirant and athlete's-foot-controlling action, in particular in the area of sport hygiene: these include antiperspirants based on aluminium or zinc salts, deodorants which contain bacteriocidal or bacteriostatic deodorizing substances, for example triclosan, hexachlorophene, alcohols and cation-active substances, for example quaternary ammonium salts and odour absorbers, for example ®Grillocin (combination of zinc ricinoleate and various additives) or triethyl citrate, optionally in combination with an antioxidant, for example butylhydroxytoluene) or ion exchange resins;

active ingredients for dental, denture and oral care which are present, for example, in toothpastes, toothpaste gels, tooth powders, mouthwash concentrates, antiplaque mouth rinses, denture cleaners or denture adhesives, for example aliphatic ammonium fluorides, in particular stearyl trihydroxyethylpropylenediamine dihydrofluoride;

active ingredients which offer protection against sunlight (UV filters): suitable active ingredients are filter substances (“sunscreens”) which are able to absorb UV radiation from sunlight and convert it into heat. Depending on the desired action, the following light protection agents are preferred: light protection agents which selectively absorb energy-rich UV radiation in the range from about 280-315 nm which produces sunburn (UV-B absorbers) and transmit the longer-wave range from about 315-400 nm (UV-A region), and light protection agents which absorb only the longer-wave radiation of the UV-A region from 315-400 nm (UV-A absorbers).

Suitable light protection agents are, for example, organic UV absorbers from the class of p-aminobenzoic acid derivatives, salicylic acid derivatives, benzophenone derivatives, dibenzoylmethane derivatives, diphenyl acrylate derivatives, benzofuran derivatives, polymeric UV absorbers comprising one or more organosilicon radicals, cinnamic acid derivatives, camphor derivatives, trianilino-s-triazine derivatives, resorcinyltriazines, phenylbenzimidazolesulfonic acid and salts thereof, menthyl anthranilates and benzotriazole derivatives.

Exemplary compounds for p-aminobenzoic acid derivatives:

4-aminobenzoic acid (PABA); ethyl dihydroxypropyl-PABA of the formula

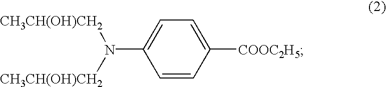

octyl dimethyl-PABA of the formula

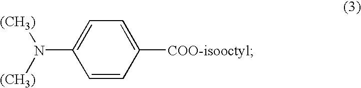

or glyceryl aminobenzoate of the formula

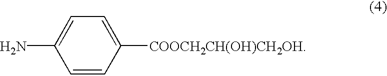

Exemplary compounds of salicylic acid derivatives:

homomenthyl salicylate of the formula

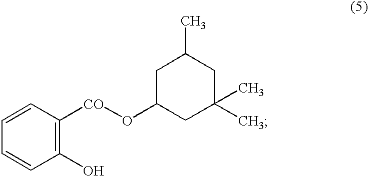

triethanolamine salicylate of the formula

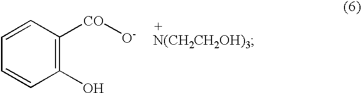

amyl p-dimethylaminobenzoate of the formula

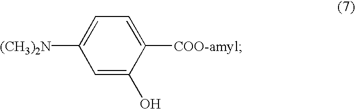

octylsalicylate of the formula

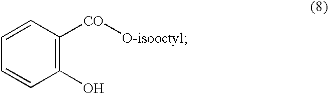

or 4-isopropylbenzyl salicylate of the formula

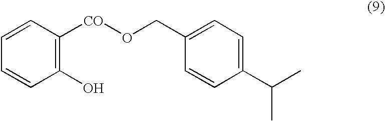

Exemplary compounds of benzophenone derivatives:

benzophenone-3-(2-hydroxy-4-methoxybenzophenone), benzophenone-4-(2-hydroxy-4-methoxybenzophenone-5-sulfonic acid) or benzophenone-8-(2,2′-dihydroxy-4-methoxybenzophenone).

Exemplary compounds of dibenzoylmethane derivatives:

butylmethoxydibenzoylmethane-[1-(4-tert-butyl)-3-(4-methoxyphenyl)propane-1,3-dione].

Exemplary compounds of diphenylacrylate derivatives:

octocrylene (2-ethylhexyl 2-cyano-3,3′-diphenylacrylate) or etocrylene (ethyl 2-cyano-3-3′-diphenylacrylate).

Exemplary compounds of benzofuran derivatives:

3-benzofuranyl 2-cyanoacrylate, 2-(2-benzofuranyl)-5-tert-butyl benzoxazole or 2-(p-aminophenyl)benzofuran and, in particular, the compound of the formula

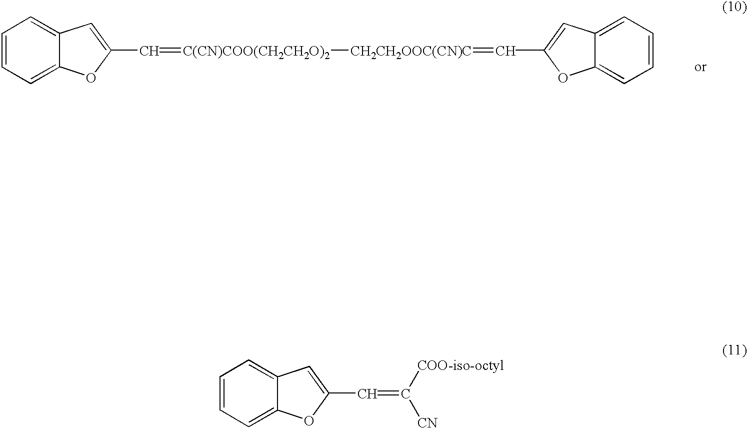

Exemplary compounds of polymeric UV absorbers which comprise one or more organosilicon radicals:

benzylidenemalonate derivative, in particular the compound of the formula

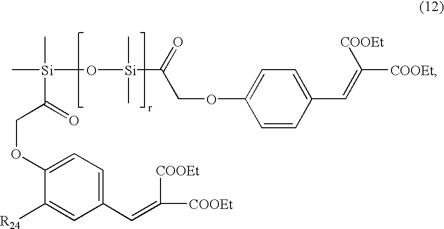

in which

R₂₄ is hydrogen or O-Me and

r is approximately 7; the compound of the formula

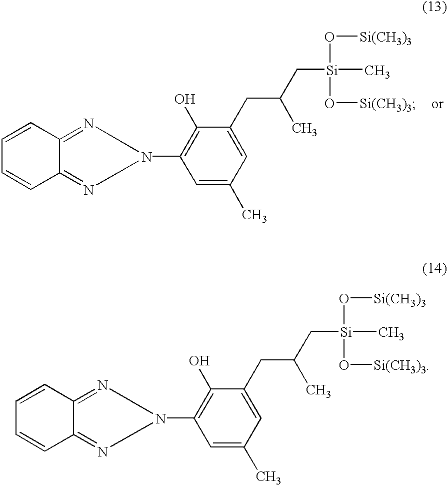

Exemplary compounds of cinnamic esters:

octyl methoxycinnamate(4-methoxycinnamic 2-ethylhexyl ester), diethanolamine methoxycinnamate (diethanolamine salt of 4-methoxycinnamic acid), isoamyl p-methoxycinnamate(4-ethoxycinnamic 2-isoamyl ester), 2,5-diisopropylmethyl cinnamate or a cinnamic acid amido derivative.

Exemplary compounds of camphor derivatives:

4-methylbenzylidenecamphor[3-(4′-methyl)benzylidenebornan-2-one], 3-benzylidenecamphor(3-benzylidenebornan-2-one), polyacrylamidomethylbenzylidenecamphor{N-[2(and 4)-2-oxyborn-3-ylidenemethyl)benzyl]acrylamide polymer}, trimoniumbenzylidenecamphor sulfate[3-(4′-trimethylammonium)benzylidenebornan-2-one methylsulfate], terephthalylidenedicamphorsulfonic acid{3,3′-(1,4-phenylenedimethine)-bis(7,7-dimethyl-2-oxobicyclo[2.2.1 ]heptane-1 -methanesulfonic acid} or salts thereof, or benzylidenecamphorsulfonic acid[3-(4′-sulfo)benzylidenebornan-2-one] or salts thereof.

Exemplary compounds of trianilino-2-triazine derivatives:

octyltriazine[2,4,6-trianilino(p-carbo-2′-ethyl-1′-oxy)-1,3,5-triazine], and the trianilino-s-triazine derivatives described in U.S. Pat. No. 5,332,568, U.S. Pat. No. 5,252,323, WO 93/17002 and WO 97/03642 and EP-A-0,517,104.

Examples of resorcinyltriazines are the compounds described in EP-A-0,775,698, in particular the compounds of the formula

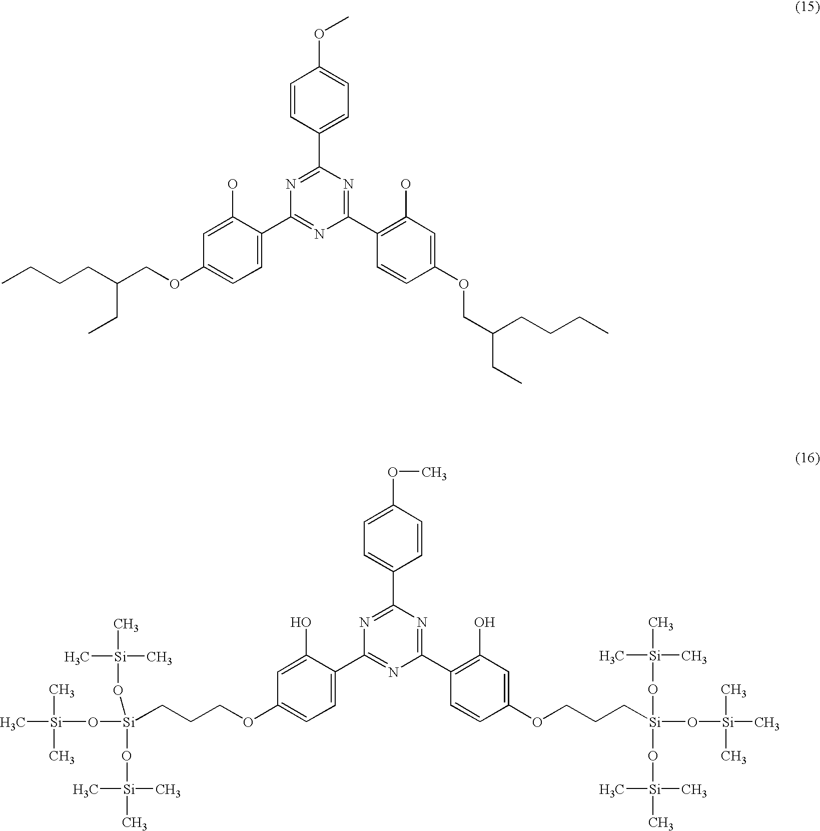

Exemplary compounds of benzotriazoles:

2-(2-hydroxy-5-methylphenyl)benzotriazole;

the benzotriazole compounds disclosed in EP-A-0,746,305, in particular the compound of the formula

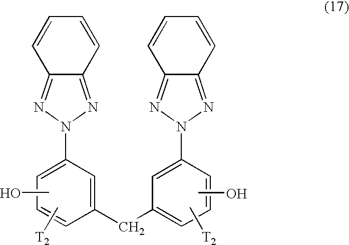

in which T₂ is hydrogen or C₁-C₈ alkyl;

active ingredients against insects (“repellants”): repellants are agents intended to prevent insects contacting with the skin and becoming active thereon. They drive away the animals and evaporate slowly. The most frequently used repellant is diethyltoluamide (DEET). Further customary repellants are given in “W. Raab and U. Kindl, “Pflegekosmetik” [Care cosmetics], Gustav-Fischer-Verlag Stuttgart/New York, 1991, p. 161.

active ingredients for protecting against chemical and mechanical effects: these include all substances which form a barrier between the skin and external noxae, for example paraffin oils, silicone oils, plant oils, PCL products and lanolin for protection against aqueous solutions, film formers, such as sodium alginate, triethanolamine alginate, polyacrylates, polyvinyl alcohol or cellulose ethers against the effect of organic solvents, or substances based on mineral oils, vegetable oils or silicone oils as “lubricants” against strong mechanical stresses of the skin;

humectant substances: the following substances are, for example, used as moisturize regulators (“moisturizers”): sodium lactate, urea, amino acids of the “natural moisturizing factor” (NMF), alcohols, sorbitol, glycerol, propylene glycol, collagen, elastin or hyaluronic acids;

active ingredients with a keratoplastic or keratolytic effect: benzoyl peroxide, retinoic acid, colloidal sulfur and resorcinol, α-hydroxycarboxylic acids, for example lactic acid, salicylic acid and derivatives thereof;

antimicrobial agents, for example halogenated diphenyl ether compounds, in particular triclosan and diclosan, diphenyl ethers, for example 4-(2-tert-butyl-5-methylphenoxy)phenyl or quaternary ammonium compounds;

agents comprising free-radical scavengers (antioxidants) or lipophilic derivatives thereof;

oily or oil-soluble vitamins or vitamin derivatives which can be applied dermally: for example vitamin A (retinol in the form of the free acid or its derivatives), panthenol, pantothenic acid, folic acid and combinations thereof, vitamin E (tocopherol), F; essential fatty acids; or niacinamide (nicotinamide) or derivatives thereof, lipophilically-derivatized water-soluble hydrophilic vitamins;

vitamin-based placenta extracts: active ingredient compositions primarily comprising vitamins A, C, E, and B₁₂, folic acid and biotin, amino acids and ferments, and compounds of the trace elements magnesium, silicon, phosphorus, calcium, manganese, iron or copper or derivatives thereof;

depigmentation agents for bleaching human hair or for lightening melanin spots (skin whitening): for example hydroquinone or derivatives thereof, ascorbic acids and derivatives and caffeic acid;

skin-tanning agents: for example dihydroxyacetone (DHA) and modifications thereof, complexing agents for the release or absorption of essential metals or lipophilically derivatized complexing agents;

skin repair complexes: obtainable from inactivated and disintegrated cultures of bacteria of the Bifidus group;

plants and plant extracts: for example arnica, aloe, beard lichen, ivy, stinging nettle, ginseng, henna, camomile, marigold, rosemary, sage, horsetail or thyme;

animal extracts, for example royal jelly, propolis, proteins or thyme extracts;

cosmetic oils which can be applied dermally: neutral oils of the Miglyol 812 type, apricot kernel oil, avocado oil, babussu oil, cottonseed oil, borage oil, thistle oil, peanut oil, gamma oryzanol, rosehip kernel oil, hemp oil, hazelnut oil, currantseed oil, jojoba oil, cherrystone oil, salmon oil, linseed oil, corn oil, macadamia nut oil, almond oil, evening primrose oil, mink oil, olive oil, pecan nut oil, peach kernel oil, pistachio kernel oil, rapeseed oil, rice kernel oil, castor oil, safflower oil, sesame oil, soybean oil, sunflower oil, teatree oil, grapeseed oil, wheatgerm oil or blue cypress oil.

The microbially encapsulatable cosmetic ingredients are preferably chosen from fragrances and cosmetic oils which can be applied dermally, from skincare and skin-cleansing substances, from active ingredients having a deodorizing and antiperspirant action, UV filters, active ingredients for dental, denture and oral care, active ingredients against insects, active ingredients for protecting against chemical and mechanical effects, humectant substances, active ingredients having a keratoplastic effect, antimicrobial agents, oily or oil-soluble vitamins or vitamin derivatives which can be applied dermally, vitamin-based placenta extracts, skin repair complexes, plants and plant extracts, animal extracts, dyes, antioxidants, skin-tanning and skin-whitening substances, and other cosmetic active ingredients.

Very particularly preferred microbially encapsulatable cosmetic ingredients are antimicrobial active ingredients chosen from 2-hydroxy-4,2′,4′-trichlorodiphenyl ether (triclosan), 2-hydroxy-4,4′-dichlorodiphenyl ether (diclosan) or 4-(2-tert-butyl-5-methylphenoxy)phenol.

The cosmetic ingredients may be present in the microbially encapsulated material as individual substances or as a mixture of two or more substances.

The microbially encapsulatable cosmetic ingredients are present in the encapsulated material in a concentration of from 1 to 80% by weight, preferably from 2 to 60% by weight, based on the total weight of the filled biocapsules.

Cosmetic end formulations include a very wide variety of cosmetic compositions. In particular, the compositions below are, for example, suitable:

skincare compositions, for example skin washes and cleansers in the form of bar or liquid soaps, syndets or washing pastes,

bath preparations, for example liquid bath preparations (foam baths, melts, shower preparations) or solid bath preparations (bath tablets or bath salts);

skincare compositions, for example skin emulsions, multiple emulsions or skin oils;

decorative body care compositions, for example face make-up in the form of day creams or powder creams, face powders (loose and pressed), blusher or cream foundation, eyecare compositions, for example eyeshadow preparations, mascara, eyeliner, eye creams or eye-fix creams; lipcare compositions, for example lipstick, lip gloss, lipliner, nailcare compositions, for example nail varnish, nail varnish remover, nail hardeners or cuticle removers;

personal hygiene compositions, for example personal hygiene washing lotions or personal hygiene sprays;

footcare compositions, for example foot baths, foot powders, foot creams or foot balsams, specifically deodorants and antiperspirants or compositions for removing calluses;

sunscreens, such as sun milks, lotions, creams oils, sunblocks or tropicals, pretanning preparations or aftersun preparations;

skin tanning compositions, for example self-tanning creams;

depigmentation compositions, for example preparations for skin bleaching or compositions for skin lightening;

insect-repellents, for example insect oils, lotions, sprays or sticks;

deodorants, such as deodorant sprays, pump sprays, and deodorant gels, sticks or roll-ons;

antiperspirants, for example antiperspirant sticks, creams or roll-ons;

compositions for cleansing and caring for bad skin, for example syndets (solid or liquid), peeling or scrub preparations or peeling masks;

depilatories in chemical form (depilation), for example depilatory powders, liquid depilatories, depilatories in cream or paste form, depilatories in gel form or aerosol foams;

shaving compositions, for example shaving soap, foaming shaving creams, nonfoaming shaving creams, foams, gels, preshave preparations for dryshaving, aftershaves or aftershave lotions;

fragrances, for example fragrance waters (eau de Cologne, eau de toilette, eau de parfum, parfum de toilette, perfume), perfume oils and perfume creams;

compositions for dental, denture and oral care, for example toothpastes, toothpaste gels, tooth powders, mouthwash concentrates, antiplaque mouth rinses, denture cleaners or denture adhesives;

cosmetic compositions for treating hair, for example hair cleansers in the form of shampoos, hair conditioners, haircare compositions, for example pretreatment compositions, hair tonic, styling creams, styling gels, pomades, hair rinses, treatment packs, intensive hair cures, compositions for shaping hair, for example waving compositions to produce permanent waves (hot wave, mild wave, cold wave), hair-smoothing preparations, liquid hair-setting compositions, hair mousses, hairsprays, bleaches, for example hydrogen peroxide solutions, lightening shampoos, bleaching creams, bleaching powders, bleaching pastes or oils, temporary, semipermanent or permanent hair colorants, preparations containing self-oxidizing dyes, or natural hair colorants, such as henna or camomile.

These listed end formulations may be in the form of very different application forms, for example

in the form of liquid preparations as an O/W emulsion,

in the form of a gel,

in the form of an oil, cream, milk or lotion,

in the form of a powder, a lacquer, a tablet or make-up,

in the form of a stick,

in the form of a spray (spray with propellant or pump spray, or an aerosol,

in the form of a foam, or

in the form of a paste.

In this connection, the liquid and semisolid application forms comprise the microbially encapsulated material in the aqueous phase, and one or more of the functional cosmetic active ingredients listed above. Solid application forms comprise the microbially encapsulated material in dehydrated form, dehydration generally being carried out by freeze drying or spray drying in the presence of customary auxiliaries.

Emulsions are heterogeneous systems which consist of two liquids (phases) which are immiscible or only miscible to a limited extent with one another. One is in the form of droplets (disperse or internal phase), while the other, being a liquid, forms a continuous phase. In the case of an O/W emulsion, the basic character of which is determined by water, oil droplets are finely dispersed within water.

Creams are usually spreadable in the range from room temperature to skin temperature, while lotions or milks are more likely to be flowable.

Gels are semisolid, more or less transparent systems in which the so-called gel former forms a three-dimensional network in which a liquid is immobilized. The clear to opaque hydrogels consist primarily of water, water-soluble substances and thickeners or gel formers. If lipids are additionally incorporated, this gives hydrodispersion gels which are slightly creamy in appearance. By contrast, oleo gels are free from water and comprise lipids as liquid components.

The cosmetic end formulation, which comprises one or more of the microbially encapsulated ingredients listed above and which can be present in the application forms mentioned above, comprises the microbially encapsulated material used according to the invention preferably in a concentration of from 0.01 to 20% by weight, preferably from 0.05 to 10% by weight, in particular 0.1 to 5% by weight.

These end formulations are further provided by the invention.

The encapsulation is generally carried out by mixing the microbial material with the liquid form of the material to be encapsulated in aqueous medium, so that the liquid forms an emulsion in the aqueous medium in order to achieve a good dispersion and to contact the microbial material. The encapsulatable material can be washed with a washed microbe or an aqueous paste or slurry of the microbe, or the encapsulatable liquid material can be mixed in a small amount of water with the dry microbes. Only small amounts of an aqueous medium are required.

If biocapsules are used for the preparation of encapsulated active ingredients in dental, denture and oral care, then β-glucan can additionally be added to the biocapsules. Thanks to the β-glucan-containing yeast capsule sheath, biocapsules achieve substantivity on the surface of the tooth and the tooth-holding apparatus (gingiva). In the case of mouth rinses or toothpastes, contact with oral bacteria or damaged enamel or tooth cement is normally only about 1 minute. Thanks to the substantivity of the capsules, the active substance has a significantly higher residence time in the dental area.

The encapsulation can be carried out at room temperature, but preferably at elevated temperature, for example from 35 to 60° C., preferably 40 to 50° C., and at least during the initial phase of the encapsulation, i.e. during the first 30 minutes.

This treatment can be continued until optimum encapsulation is achieved.

The cosmetic end formulation according to the invention can also comprise further components, for example emollients, emulsion stabilizers, skin moisturizers, skin-tanning accelerators, thickeners, for example xanthan, moisture-retention agents, for example glycerol, preservatives, for example paraben, antioxidants, fragrances and dyes.

The end formulations are prepared by the customary known methods.

The cosmetic end formulation is preferably used for the care and protection of the skin, mucous membrane, oral cavity, nails or hair, and very particularly has a sunscreen, so-called “aftersun” preparation, aftershave preparation, eau de toilette or deodorant.

In addition, the biocapsules used according to the invention can also be used as transportation vehicles for oil-soluble dyes.

The invention therefore further provides for the use of the microbially encapsulated materials defined in claim 1 as a carrier system for oil-soluble dyes.

In this connection, suitable dyes are those both of synthetic or natural origin which are constructed from all known chromophores, for example azo, azoic, anthraquinone, carotenoid, quinoline, xanthene, diarylmethane, triarylmethane, stilbene, indigoid, phthalocyanine, nitro dyes, and all other known chromophores, as also listed in the Colour Index under CI 11000 to CI 77999.

Of these, those which are soluble at least to a small extent in organic media, such as oils, are of particular interest. These include, for example, the dyes referred to as solvent dyes or disperse dyes, the disperse dyes also including the group of uncharged directly striking hair dyes, e.g. derivatives of nitrobenzene or nitrodiphenylamine.

Examples of solvent dyes which can be used according to the invention are:

Solvent Black 3 (CAS No.: 4197-25-5); Solvent Black 5 (CAS No.: 11099-03-9); Solvent Blue 35 (CAS No.: 12769-17-4); Solvent Green 3; Solvent Green 7 (CAS No.: 6358-69-6); Solvent Orange 1 (CAS No.: 2051-85-6); Solvent Red 24 (CAS No.: 85-83-6); Solvent Red 43 (CAS No.: 15086-94-9); Solvent Red 48 (CAS No.: 13473-26-2); Solvent Red 49:1 (CAS No.: 6373-07-5); Solvent Red 72 (CAS No.: 596-03-2); Solvent Yellow 44 (CAS No.: 2478-20-8); Solvent Yellow 18 (CAS No.: 6407-76-9).

Examples of disperse dyes which can be used according to the invention are:

Disperse Black 9 (CAS No.: 12222-60-4); Disperse Blue 1 (CAS No.: 2475-45-8); Disperse Blue 3 (CAS No.: 2475-46-9); Disperse Brown 1 (CAS No.: 23355-64-8); Disperse Orange 3 (CAS No.: 730-40-5); compound of the formula

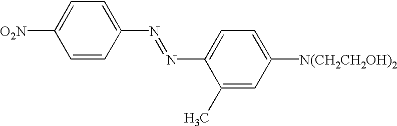

Disperse Violet 1 (CAS No.: 128-95-0); Disperse Violet 4 (CAS No.: 1220-94-6); compound of the formula

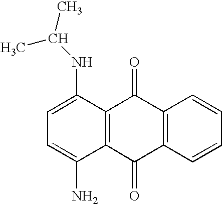

Examples of (uncharged) direct-striking hair dyes:

HC Blue No. 2 (CAS No.: 33229-34-4); HC Blue No. 4 (reaction product of N-methyl-1,4-diaminoanthraquinone, epichlorohydrin and monoethanolamine); HC Blue No. 5 (CAS No.: 68478-64-8); compound of the formula

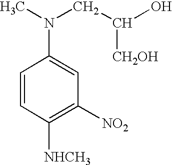

HC Blue No. 7 (CAS No.: 90817-34-8); HC Blue No. 8 (CAS No.: 22366-99-0); HC Blue No. 9 (CAS No.: 114087-42-2); HC Blue No. 10 (CAS No.: 102767-27-1); HC Blue No. 11 (CAS No.: 23920-15-2); HC Blue No. 12 (CAS No.: 132885-85-9); compound of the formula

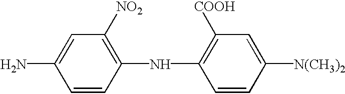

HC Blue No. 14 (CAS No.: 99788-75-7); HC Orange No. 1 (CAS No.: 54381-08-7); HC Orange No. 2 (CAS No.: 85765-48-6), HC Orange No. 3 (CAS No.: 81612-54-6); compound of the formula

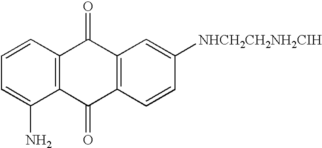

HC Red No. 1 (CAS No.: 2784-89-6); HC Red No. 3 (CAS No.: 2871-01-4); HC Red No. 7 (CAS No.: 24905-87-1); HC Red No. 8 (CAS No.: 13556-29-1); HC Red No. 9 (CAS No.: 56330-88-2); HC Red No. 10 (CAS No.: 95576-89-9); HC Red No. 11 (CAS No.: 95576-92-4); HC Red No. 13 (CAS No.: 94158-13-1); compound of the formula

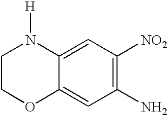

HC Violet No. 1 (CAS No. 82576-75-8); compound of the formula

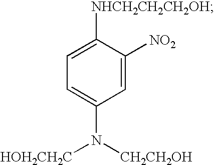

HC Yellow No. 2 (CAS No. 4926-55-0); HC Yellow No. 4 (CAS No.: 59820-43-8); HC Yellow No. 5 (CAS No.: 56932-44-6); HC Yellow No. 6 (CAS No.: 104335-00-6); compound of the formula

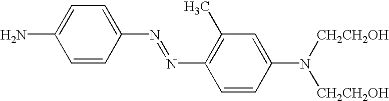

HC Yellow No. 8 (CAS No.: 66612-11-1); HC Yellow No. 9 (CAS No.: 86419-69-4); HC Yellow No. 10 (CAS No.: 109023-83-8); HC Yellow No. 11 (CAS No.: 73388-54-2); HC Yellow No. 12 (CAS No.: 59320-13-7); HC Yellow No. 13 (CAS No.: 10442-83-8); compound of the formula

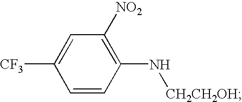

compound of the formula

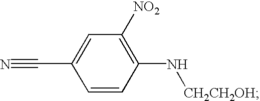

compound of the formula

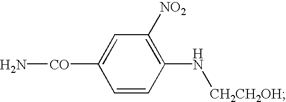

Brown No. 1 (CAS No.: 83803-98-9); HC Brown No. 2 (CAS No.: 83803-99-0); HC Green No. 1 (CAS No.: 52136-25-1).

The biocapsules used according to the invention can have the following functions:

as carrier to allow dyes to penetrate through certain potential walls into media such as the skin, hair or nails;

as protection for the dyes against other ingredients of a formulation (or vice versa, as a protection for the formulation against another dye) in order, for example, to eliminate incompatibilities between formulation and dye. The dye is then within the nanodispersion, separate from the remaining formulation. As a result, it is, for example, possible to prevent chain reactions photoinitiated by the dye with other ingredients;

in order to incorporate and stabilize dyes that are only soluble in oil phases in aqueous systems.

In the examples below, the percentages are by weight. Unless stated otherwise, in the case of the compounds used, the amounts are based on the pure substance.

Preparation of Microbially Encapsulated Cosmetic Ingredients (Filled Biocapsules)

### EXAMPLE 1

Pressed baker's yeast is slurried in distilled water, washed several times with distilled water, and, using a Biocentrifuge 28RS (Heraeus) at 17,000 rpm, separated off from the aqueous phase. 75 g (dry weight 17.4 g) of this solution are mixed for 5 h at 45° C. with an amount of teatree oil corresponding to the dry weight, and the excess teatree oil is removed by absorption. The resulting product comprises 63% encapsulated teatree oil.

### EXAMPLE 2

A 25 l culture of S. cerevisiae is concentrated by centrifugation and washed a number of times with distilled water. The yeast cake weighing 212 g (37.2 g dry weight) is mixed with 40 ml of a 5% methanolic solution of retinol under nitrogen over the course of 4 h at 35° C. The yeast capsules are then dried and stored under a nitrogen atmosphere until formulation. The yeast capsules show a retinol content of 2.5%.

### EXAMPLE 3

20 g of a washed baker's yeast with a dry weight of 4.1 g are mixed with a mixture of 2 g of ascorbic acid and 2 g of tocopherol in ethanol over the course of 6 h at 40° C. The dried yeast capsules contain a total of 56% of the ascorbic acid/tocopherol mixture.

### EXAMPLE 4

20 g of a washed brewer's yeast with a dry weight of 3.9 g are mixed with 4 g of Blue Cypress Oil over the course of 5 h at 40° C. The dried yeast capsules, after trituration, intensively exude the typical odour of Blue Cypress Oil. They contain 61% of the oil.

### EXAMPLE 5

20 g of a washed baker's yeast with a dry weight of 4.1 g are mixed with 4 g of tocopherol acetate over the course of 5 h at 40° C. The dried yeast capsules contain a total of 65% of tocopherol acetate.

Examples of Cosmetic End Formulations

### EXAMPLE 6

### Sun Cream Containing Vitamin A

Composition:

| | | |
|---|---|---|
| (1) | Water | to 100 |
| (2) | Biocapsules according to Example 2 | 1.00 |
| (3) | Sodium acrylates copolymer and soybean oil and PPG-1 | 2.50 |
| | trideceth-6 Octyl methoxycinnamate | |
| (4) | d-Panthenol | 6.50 |
| (5) | Imidazolidinyl urea | 0.50 |
| (6) | Phenoxyethanol (and) methylparaben (and) butylparaben | 0.20 |
| | (and) | |
| (7) | ethylparaben (and) propylparaben | 0.30 |
| | Aloe Barbadensis | |
| (8) | Fragrance | 1.00 |
| (9) | Alcohol denat. | 0.35 |
| (10) | Propylene glycol | 8.50 |
| (11) | | 3.00 |

Preparation

a. (1) is weighed into a clean, dry beaker; all ingredients apart from (2) are added one after the other. After each addition of a further component, the mixture is stirred.

b. The stirring speed is increased and (2) is added with continuous stirring until a smooth and viscous solution is formed.

### EXAMPLE 7

### Foot Cream Containing Blue Cypress Oil

Composition

| | | |
|---|---|---|
| (1) | Water | to 100 |
| (2) | Biocapsules according to Example 4 | 2.00 |
| (3) | Sodium acrylates copolymer and mineral oil and PPG-1 | 3.00 |
| | trideceth-6 Buxus Chinensis | |
| (4) | Persea Gratissima | 5.00 |
| (5) | Dimethicone (and) dimethiconol | 5.00 |
| (6) | Sodium methylparaben (and) sodium butylparaben (and) | 10.00 |
| | sodium | |
| (7) | ethylparaben (and) sodium propylparaben | 0.10 |
| (8) | Benzophenone-4 | 0.15 |
| (9) | Fragrance | 0.10 |
| (10) | Colour(s) | qs |

Preparation

a. (1) is weighed into a clean, dry beaker and stirring is started.

b. (6), (7), (8) and (9) are added and the mixture is stirred.

c. All components, apart from (2) are added in the appropriate order with stirring.

d. The stirring speed is increased and (2) is added; the mixture is stirred further until the composition is homogeneous.

### EXAMPLE 8

### Mouthwash Containing Teatree Oil

Composition (100% Active Substances)

| | | |
|---|---|---|
| (A) | Triclosan | 0.05% |
| (B) | Glycerol | 10.0% |
| | Ethanol | 5.0% |
| (C) | Poloxamer 407 | 2.0% |
| (D) | Sodium saccharin | 0.125% |
| | Sodium fluoride | 0.5% |
| | Sodium benzoate | 0% |
| | Methylparaben | 0.2% |
| | Propylparaben | 0.3% |
| (E) | Deionized water | to 100.0% |
| | Capsules according to 1.1 | 1.0% |
| | Flavour | qs |

Preparation:

(A) is dissolved in the mixture of (B).

(C) is dissolved in 25% of (E) by heating to 70° C.

The compounds (D) are dissolved in the remainder of E. The solution (A)-(B) is added to the solution (C)-(D).

### EXAMPLE 9

### Hair and Body Shampoo Containing Blue Cypress Oil

Composition (100% Active Substances)

| | | |
|---|---|---|
| (A) | Sodium laureth sulfate | 7.8% |
| | Disodium laureth sulfosuccinate | 5.2% |
| | PEG-7 Glyceryl cocoate | 4.0% |
| | PEG-3 distearate (and) sodium laureth sulfate | 3.0% |
| | Laureth-2 | 1.2% |
| | Sodium chloride | 3.0% |
| | Dye, perfume and preservative | q.s. |
| (B) | Biocapsules according to Example (4) | 2% |
| | Deionized water up to | 100% |

Preparation

(A) is mixed with 10% water until a homogeneous solution is formed. (B) is used to make up the solution to 100%.

### EXAMPLE 10

### Aftershave Cream Containing Vitamin C and Vitamin E

Composition

| | | |
|---|---|---|
| (1) | Water | to 100 |
| (2) | Polyquaternium-37 and mineral oil and PPG-1 | 2.85% |
| | trideceth-6 Alcohol denat. | |
| (3) | Isopropyl palmitate | 7.50% |
| (4) | Caprylic/capric triglycerides | 5.00% |
| (5) | Glycerol | 5.00% |
| (6) | Biocapsules according to Example 3 | 4.00% |
| (7) | Cyclomethicone | 0.80% |
| (8) | Aloe Barbadensis | 2.00% |
| (9) | Fragrance | 0.20% |
| (10) | Octyl triazone | 0.10% |
| (11) | Sodium methylparaben (and) sodium butylparaben (and) | 0.15% |
| | sodium | |
| (12) | ethylparaben (and) sodium propylparaben | 0.10% |
| (13) | Colour(s) | qs |

Preparation

a. (1) is weighed into a clean, dry beaker and stirring is started.

b. (11), (12), (10) and (13) are dispersed.

c. All ingredients apart from (2) are added one after the other; in between each one being added the mixture is stirred.

d. The stirrer speed is increased; (2) is added and the mixture is stirred until the product is viscous and homogeneous.

### EXAMPLE 11

### Preparation of Microbially Encapsulated Fluorides

100 g of a washed baker's yeast with a dry weight of 19.3 g are suspended in an ethanolic solution comprising 10% stearyl trihydroxyethylpropylenediamine dihydrofluoride (olaflur) and incubated at 35° C. for 6 h. Following suction filtration and drying, the yeast capsules have a stearyl trihydroxyethylpropylenediamine dihydrofluoride content of 22%.

### EXAMPLE 12

### Preparation of Microbially Encapsulated 4-(2-tert-butyl-5-methylphenoxy)phenol

100 g of a washed baker's yeast with a dry weight of 18.9 g are suspended in a 40% ethanolic solution containing 4-(2-tert-butyl-5-methylphenoxy)phenol and incubated at 45° C. for 4 h. Following suction filtration and drying, the yeast capsules have a 4-(2-tert-butyl-5-methylphenoxy)phenol content of 35%.

A comparable loading of the capsules (33%) with 4-(2-tert-butyl-5-methylphenoxy)phenol was obtained using a 30% 4-(2-tert-butyl-5-methylphenoxy)phenol solution in 2-butyl-1-octanol and incubation for 3½ h at 50C.

### EXAMPLE 13

### Preparation of Microbially Encapsulated Triclosan

100 g of a washed baker's yeast with a dry weight of 19.5 g are suspended in a 25% strength ethanolic solution of triclosan and incubated at 45° C. for 5 h. After suction filtration and drying, the yeast capsules have a triclosan content of 28%.

### EXAMPLE 14

### Preparation of a Toothpaste Containing 4-(2-tert-butyl-5-methylphenoxy)phenol

| Ingredients | Percentages by weight |
|---|---|
| Distilled water | to 100 |
| Sorbitol | 34.50 |
| Zeodent 113 | 20 |
| Glycerol | 20 |
| Tetrasodium pyrophosphate | 12.0 |
| Disodium pyrophosphate | 3.40 |
| Sodium lauryl phosphate | 1.37 |
| Flavours | 1.35 |
| PEG-6 | 1.33 |
| Aqualon CMC-9M31XF | 1.00 |
| Biocapsules prepared according to Example 12 | 0.5 |
| Sodium fluoride | 0.50 |
| Carbomer 940 | 0.20 |
| Saccharin-sodium | 0.20 |
| Titanium dioxide | 0.16 |
| P-chitosan | 0.03 |
| FD & C Blue (No. 1, 1% soln.) | 0.03 |

### EXAMPLE 15

### Preparation of a Toothpaste Containing Encapsulated Alkylammonium Fluoride

| Ingredients | Percentages by weight |
|---|---|
| Distilled water | to 100 |
| Sorbitol | 34.25 |
| Zeodent 113 | 20 |
| Glycerol | 20 |
| Tetrasodium pyrophosphate | 12.0 |
| Disodium pyrophosphate | 3.40 |
| Sodium lauryl phosphate | 1.37 |
| Flavours | 1.35 |
| PEG-6 | 1.33 |
| Aqualon CMC-9M31XF | 1.00 |
| Biocapsules, prepared according to Example 12 | 0.75 |
| Sodium fluoride | 0.50 |
| Carbomer 940 | 0.20 |
| Saccharin-sodium | 0.20 |
| Titanium dioxide | 0.16 |
| P-chitosan | 0.03 |
| FD & C Blue (No. 1, 1% soln.) | 0.03 |

### EXAMPLE 16

### Preparation of a Toothpaste Contaning Encapsulated 4-(2-tert-butyl-5-methylphenoxy)phenol and Slow Release Fluorine-encapsulated Alkylammonium Fluoride

| Ingredients | Percentages by weight |
|---|---|
| Distilled water | to 100 |
| Sorbitol | 34.50 |
| Zeodent 113 | 20 |
| Glycerol | 20 |
| Tetrasodium pyrophosphate | 12.0 |
| Disodium pyrophosphate | 3.40 |
| Sodium lauryl phosphate | 1.37 |
| Flavours | 1.35 |
| PEG-6 | 1.33 |
| Aqualon CMC-9M31XF | 1.00 |
| Biocapsules, prepared according to Example 12 | 0.5 |
| Biocapsules, prepared according to Example 11 | 0.75 |
| Sodium fluoride | 0.50 |
| Carbomer 940 | 0.20 |
| Saccharin-sodium | 0.20 |
| Titanium dioxide | 0.16 |
| P-chitosan | 0.03 |
| FD & C Blue (No. 1, 1% soln.) | 0.03 |

### EXAMPLE 17

### Preparation of a Toothpaste Containing Encapsulated Triclosan and Slow Release Fluorine-encapsulated Alkylammonium Fluoride

| Ingredients | Percentages by weight |
|---|---|
| Distilled water | to 100 |
| Sorbitol | 34.50 |
| Zeodent 113 | 20 |
| Glycerol | 20 |
| Tetrasodium pyrophosphate | 12.0 |
| Disodium pyrophosphate | 3.40 |
| Sodium lauryl phosphate | 1.37 |
| Flavours | 1.35 |
| PEG-6 | 1.33 |
| Aqualon CMC-9M31XF | 1.00 |
| Biocapsules, prepared according to Example 13 | 5 |
| Biocapsules, prepared according to Example 11 | 0.75 |
| Sodium fluoride | 0.50 |
| Carbomer 940 | 0.20 |
| Saccharin-sodium | 0.20 |
| Titanium dioxide | 0.16 |
| P-chitosan | 0.03 |
| FD & C Blue (No. 1, 1% soln.) | 0.03 |

### EXAMPLE 18

### Preparation of a Mouthwash Containing Encapsulated 4-(2-tert-butyl-5-methyllphenoxy)phenol

| Ingredients | Percentages by weight |
|---|---|
| Distilled water | to 100 |
| Ethanol | 10 |
| Glycerol | 10 |
| Pluronic F108 | 2 |
| Tetrasodium pyrophosphate | 1.5 |
| Flavours | 1.35 |
| Disodium pyrophosphate | 0.5 |
| Biocapsules, prepared according to Example 12 | 0.5 |
| Sodium fluoride | 0.5 |
| Saccharin-Na | 0.3 |
| P-chitosan | 0.02 |

### EXAMPLE 19

### Preparation of a Mouthwash Containing Encapsulated Triclosan

| Ingredients | Percentages by weight |
|---|---|
| Distilled water | to 100 |
| Ethanol | 10 |
| Glycerol | 10 |
| Pluronic F108 | 2 |
| Tetrasodium pyrophosphate | 1.5 |
| Flavours | 1.35 |
| Biocapsules, prepared according to Example 13 | 0.5 |
| Disodium pyrophosphate | 0.5 |
| Sodium fluoride | 0.5 |
| Saccharin-Na | 0.3 |
| P-chitosan | 0.02 |

### EXAMPLE 20

### Preparation of a Mouthwash Containing Fluorine Biocapsules

| Ingredients | Percentages by weight |
|---|---|
| Distilled water | to 100 |
| Ethanol | 10 |
| Glycerol | 10 |
| Pluronic F108 | 2 |
| Tetrasodium pyrophosphate | 1.5 |
| Flavours | 1.35 |
| Biocapsules, prepared according to Example 11 | 0.75 |
| Disodium pyrophosphate | 0.5 |
| Sodium fluoride | 0.5 |
| Saccharin-Na | 0.3 |
| P-chitosan | 0.02 |

### EXAMPLE 21

### Preparation of a Mouthwash Containing 4-(2-tert-butyl-5-methylphenoxy)phenol and alkyl fluoride

| Ingredients | Percentages by weight |
|---|---|
| Distilled water | to 100 |
| Ethanol | 10 |
| Glycerol | 10 |
| Pluronic F108 | 2 |
| Tetrasodium pyrophosphate | 1.5 |
| Flavours | 1.35 |
| Biocapsules, prepared according to Example 11 | 0.75 |
| Biocapsules prepared according to Example 12 | 0.50 |
| Disodium pyrophosphate | 0.5 |
| Sodium fluoride | 0.5 |
| Saccharin-Na | 0.3 |
| P-chitosan | 0.02 |

### EXAMPLE 22

### Foot Cream Containing Microbially Encapsulated 4-(2-tert-butyl-5-methylphenoxy)phenol

| | | |
|---|---|---|
| (1) | Water | to 100 |
| (2) | Biocapsules prepared according to Example 12 | 2.00 |
| (3) | Sodium acrylates copolymer and mineral oil and | 3.00 |
| | PPG-1 trideceth-6 | |
| (4) | Buxus Chinensis | 5.00 |
| (5) | Persea Gratissima | 5.00 |
| (6) | Dimethicone (and) dimethiconol | 10.00 |
| (7) | Sodium methylparaben (and) sodium butylparaben | 0.10 |
| | (and) sodium ethylparaben (and) sodium propylparaben | |
| (8) | Benzophenone-4 | 0.15 |
| (9) | Fragrance | 0.10 |
| (10) | Colour(s) | qs |

### EXAMPLE 23

### Foot Cream Containing Microbially Encapsulated 4-(2-tert-butyl-5-methylphenoxy)phenol (Double-active)

| | | |
|---|---|---|
| (1) | Water | to 100 |
| (2) | Biocapsules according to Example 4 | |
| (3) | Biocapsules prepared according to Example 12 | 2.00 |
| (4) | Sodium acrylates copolymer and mineral oil and | 3.00 |
| | PPG-1 trideceth-6 | |
| (5) | Buxus Chinensis | 5.00 |
| (6) | Persea Gratissima | 5.00 |
| (7) | Dimethicone (and) dimethiconol | 10.00 |
| (8) | Sodium methylparaben (and) sodium butylparaben (and) | 0.10 |
| | sodium ethylparaben (and) sodium propylparaben | |
| (9) | Benzophenone-4 | 0.15 |
| (10) | Fragrance | 0.10 |
| (11) | Colour(s) | qs |

What is claimed is:

1. The use of microbially encapsulated materials (filled biocapsules) in cosmetic end formulations, wherein the biocapsules comprise one or more liquid or microbially encapsulatable cosmetic ingredients.

2. The use according to claim 1, wherein the biocapsules consist of yeast.

3. The use according to claim 1 or 2, wherein the microbially encapsulatable cosmetic ingredients are chosen from fragrances and cosmetic oils which can be applied dermally, from skincare and skin-cleansing substances, from active ingredients having a deodorizing and antiperspirant action, UV filters, active ingredients against insects, active ingredients for protecting against chemical and mechanical effects, humectant substances, active ingredients having a keratoplastic or keratolytic effect, antimicrobial agents, oily or oil-soluble vitamins or vitamin derivatives which can be applied dermally, vitamin-based placenta extracts, skin repair complexes, plants and plant extracts, animal extracts, dyes, antioxidants, skin-tanning and skin-whitening substances, and other cosmetic active ingredients.

4. The use according to claim 3, wherein the microbially encapsulatable cosmetic ingredients are chosen from triclosan, diclosan or 4-(2-tert-butyl-5-methylphenoxy)phenol.

5. The use according to any one of claims 1 to 4, wherein the cosmetic ingredients are present in the microbially encapsulated material as individual substances or as a mixture of two or more substances.

6. The use according to any one of claims 1 to 5, wherein the microbially encapsulatable cosmetic ingredients are present in the encapsulated material in a concentration of from 1 to 80% by weight, based on the total weight of the filled biocapsules.

7. The use according to any one of claims 1 to 6, wherein the microbially encapsulated ingredients are each present in the cosmetic end formulation in a concentration of from 0.01 to 20% by weight, based on the cosmetic end formulation.

8. A cosmetic end formulation in the form of a gel, comprising the microbially encapsulated material as defined in claim 1.

9. A cosmetic end formulation in the form of a cream, a lotion or a milk, comprising the microbially encapsulated material as defined in claim 1.

10. A cosmetic end formulation in the form of a stick, comprising the microbially encapsulated material as defined in claim 1.

11. A cosmetic end formulation in the form of a spray or aerosol, comprising the microbially encapsulated material as defined in claim 1.

12. A cosmetic end formulation in the form of a foam, comprising the microbially encapsulated material as defined in claim 1.

13. A cosmetic end formulation in the form of a paste, comprising the microbially encapsulated material as defined in claim 1.

14. A cosmetic end formulation in the form of a powder, a varnish, a tablet or make-up, comprising the microbially encapsulated material as defined in claim 1.

15. The use of the cosmetic end formulation according to any one of claims 8 to 14 for the care and protection of the skin, mucous membrane, oral cavities, hair or nails.

16. The use of the cosmetic end formulation according to any one of claims 8 to 14 as a sunscreen or as an “aftersun” preparation.

17. The use of the cosmetic end formulation according to any one of claims 8 to 14 for shaving or as an “aftershave” preparation.

18. The use of the cosmetic end formulation according to any one of claims 8 to 14 as perfume, eau de toilette or deodorant.
